(19) United States
(12) Patent Application Publication
(10) Pub. No.: US 2015/0305697 A1
(43) Pub. Date: Oct. 29, 2015

(54) X-RAY COMPUTED TOMOGRAPHY APPARATUS AND PHOTON COUNTING CT APPARATUS

(71) Applicants: KABUSHIKI KAISHA TOSHIBA, Minato-ku (JP); Toshiba Medical Systems Corporation, Otawara-shi (JP)

(72) Inventors: Emi Tamura, Nasushiobara (JP); Yasuo Saito, Nasushiobara (JP); Takuzo Takayama, Utsunomiya (JP); Hiroaki Miyazaki, Otawara (JP); Hiroaki Nakai, Nasushiobara (JP)

(73) Assignees: KABUSHIKI KAISHA TOSHIBA, Minato-ku (JP); Toshiba Medical Systems Corporation, Otawara-shi (JP)

(21) Appl. No.: 14/740,692

(22) Filed: Jun. 16, 2015

Related U.S. Application Data
(63) Continuation of application No. PCT/JP2015/050147, filed on Jan. 6, 2015.

(30) Foreign Application Priority Data
Jan. 6, 2014 (JP) 2014-000592

(51) Int. Cl. A61B 6/00; A61B 6/06; H05G 1/10; A61B 6/03

(52) U.S. Cl. CPC A61B 6/4007; A61B 6/035; A61B 6/4241; A61B 6/5205; A61B 6/4035; A61B 6/06; H05G 1/10

(57) ABSTRACT

A gantry includes an X-ray source ring and a detector ring. The X-ray source ring includes a plurality of X-ray sources arrayed circumferentially. The detector ring is provided next to the X-ray source ring and includes a plurality of X-ray detectors arrayed circumferentially. Each of the plurality of X-ray detectors detects X-rays from the X-ray source ring. At least one wedge filter is provided on the inner circumferential side of the X-ray source ring. A filter support member supports at least one wedge filter rotatable about the rotation axis. A filter driver drives the filter support member. An imaging controller controls the filter driver to rotate at least one wedge filter about the rotation axis in synchronism with generation of X-rays from the X-ray source ring.

### CROSS-REFERENCE TO RELATED APPLICATIONS

This application is a Continuation application of PCT Application No. PCT/JP2015/050147, filed Jan. 6, 2015 and based upon and claims the benefit of priority from the Japanese Patent Application No. 2014-000592, filed Jan. 6, 2014 the entire contents of which are incorporated herein by reference.

### FIELD

Embodiments described herein relate generally to an X-ray computed tomography apparatus and a photon counting CT apparatus.

### BACKGROUND

In third-generation CT, raw data is collected by rotating a rotating ring equipped with one or more sets of X-ray tubes and X-ray detectors. The rotating ring has reached the highest rotational speed of 0.275 s/rot. In physics, the centrifugal force generated by rotation is proportional to the square of angular velocity. For this reason, it is difficult to greatly increase the current rotational speed of the rotating ring. In fifth-generation CT, an electron gun is used to emit an electron beam from the rear side of a gantry, and the electron path is deflected by using a coil to cause the electron beam to strike anodes arrayed on a circumference, thereby generating X-rays. An electron beam is deflected onto the circumference to implement CT. In fifth-generation CT, since X-ray detectors are arrayed on a circumference, the scan time is determined by the electron beam scan time. The scan time according to the fifth-generation CT has reached 50 ms to 100 ms.

U.S. Pat. No. 7,634,045 has proposed a scheme of rotating only the detector side collimator (post-collimator) mounted on a gantry in fifth-generation CT. U.S. Pat. No. 7,634,045 has also presented fifth-generation CT which can also cope with spectral CT by changing an applied voltage for each place. This scheme, however, uses an electron gun, and hence the overall size of the system becomes large. In addition, since an X-ray detector and an electron beam are offset from each other in terms of a positional relationship, this scheme is not suitable for three-dimensional scanning (volume scanning).

### BRIEF DESCRIPTION OF THE DRAWING

FIG. 1 is a block diagram showing the arrangement of an X-ray computed tomography apparatus according to the first embodiment.

FIG. 2 is a view schematically showing the structure of a gantry in FIG. 1.

FIG. 3 is a schematic front view of an X-ray source ring in FIG. 2.

FIG. 4 is a schematic front view of a detector ring in FIG. 2.

FIG. 5 is a view schematically showing the structure of an X-ray source in FIG. 1.

FIG. 6 view schematically showing the structure of another X-ray source different from the X-ray source in FIG. 5.

FIG. 7 is a longitudinal sectional view of a gantry according this embodiment.

FIG. 8 is a longitudinal sectional view showing the detailed structure of the X-ray source ring in FIG. 7.

FIG. 9A is a plan view showing the placement of an X-ray source, a wedge filter, and a post-collimator at time t when the number of X-ray sources simultaneously driven is one according to the first embodiment.

FIG. 9B is a plan view showing the placement of the X-ray source, the wedge filter, and the post-collimator at time t+Δt when the number of X-ray sources simultaneously driven is one according to the first embodiment.

FIG. 10A is a plan view showing the placement of X-ray sources, wedge filters, and post-collimators at time t when the number of X-ray sources simultaneously driven is four according to the first embodiment.

FIG. 10B is a plan view showing the placement of the X-ray sources, the wedge filters, and the post-collimators at time t+Δt when the number of X-ray sources simultaneously driven is four according to the first embodiment.

FIG. 11 is a graph schematically showing the energy spectra of X-rays generated from an X-ray source upon application of different tube voltages according to an application example of the first embodiment.

FIG. 12A is a plan view showing the placement of the X-ray sources, the wedge filters, and the post-collimators at time t when the number of X-ray sources simultaneously driven is three in tube-voltage-based spectral CT according to the application example of the first embodiment.

FIG. 12B is a plan view showing the placement of the X-ray sources, the wedge filters, and the post-collimators at time t+Δt when the number of X-ray sources simultaneously driven is three in tube-voltage-based spectral CT according to the application example of the first embodiment.

FIG. 13 is a graph schematically showing the energy spectra of X-rays emitted from an X-ray source and transmitted through wedge filters with different X-ray attenuation coefficients according to the application example of the first embodiment.

FIG. 14A is a plan view showing the placement of the X-ray sources, the wedge filters, and the post-collimators at time t when the number of X-ray sources simultaneously driven is three in filter-based spectral CT according to the application example of the first embodiment.

FIG. 14B is a plan view showing the placement of the X-ray sources, the wedge filters, and the post-collimators at time t+Δt when the number of X-ray sources simultaneously driven is three in filter-based spectral CT according to the application example of the first embodiment.

FIG. 15 is a block diagram showing the arrangement of a photon counting CT apparatus according to the second embodiment.

### DETAILED DESCRIPTION

In general, according to one embodiment, An X-ray computed tomography apparatus includes an X-ray source ring, an X-ray detector, a filter support mechanism, a filter driver, a controller, a data collection unit, and a reconstruction unit. The X-ray source ring includes a plurality of X-ray sources arrayed circumferentially. The plurality of X-ray sources individually generates X-rays. The X-ray detector detects X-rays from the X-ray source ring. The filter support mechanism supports at least one wedge filter rotatable about a rotation axis. The at least one wedge filter is provided on an inner circumferential side of the X-ray source ring. The filter driver drives the filter support mechanism. The controller controls the filter driver to rotate the at least one wedge filter about the rotation axis in synchronism with generation of X-rays from the plurality of X-ray sources. The data collection unit collects digital data corresponding to an intensity of the detected X-rays. The reconstruction unit reconstructs a CT image based on the digital data.

An X-ray computed tomography apparatus and a photon counting CT apparatus according to an embodiment will be described below with reference to the accompanying drawings.

### First Embodiment

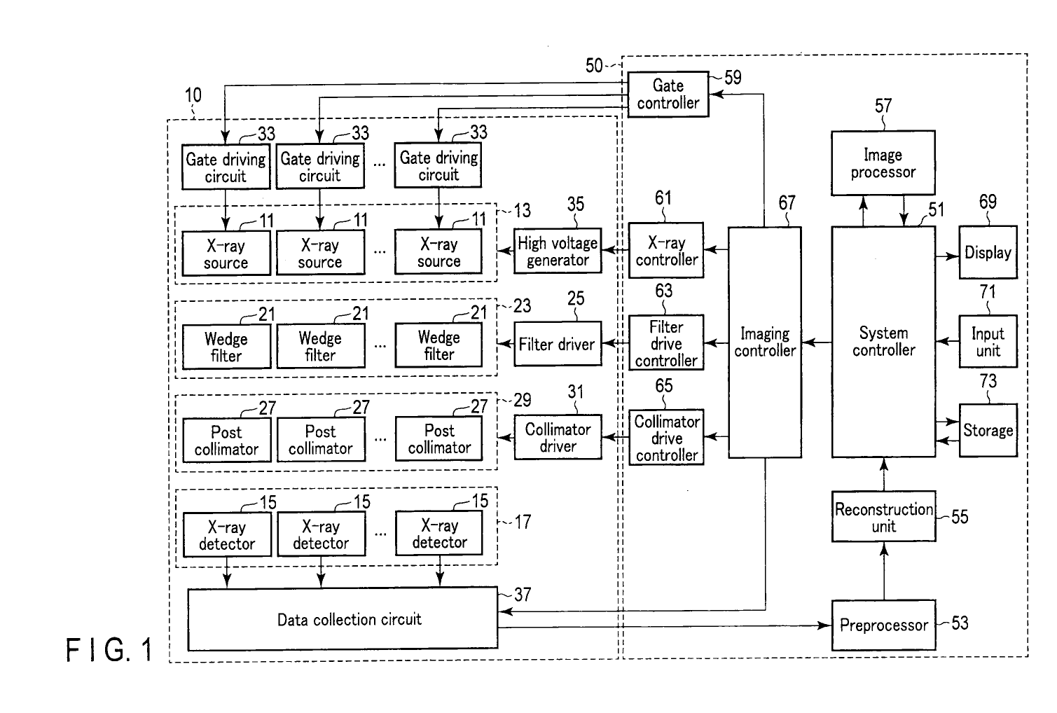

FIG. 1 shows the arrangement of an X-ray computed tomography apparatus according to the first embodiment. As shown in FIG. 1, the X-ray computed tomography apparatus according to the first embodiment includes a gantry 10 and a console 50. The gantry 10 is installed in, for example, a CT imaging room. The console 50 is installed, for example, in an imaging control room adjacent to the CT imaging room. The gantry 10 and the console 50 are communicably connected to each other via a network or the like.

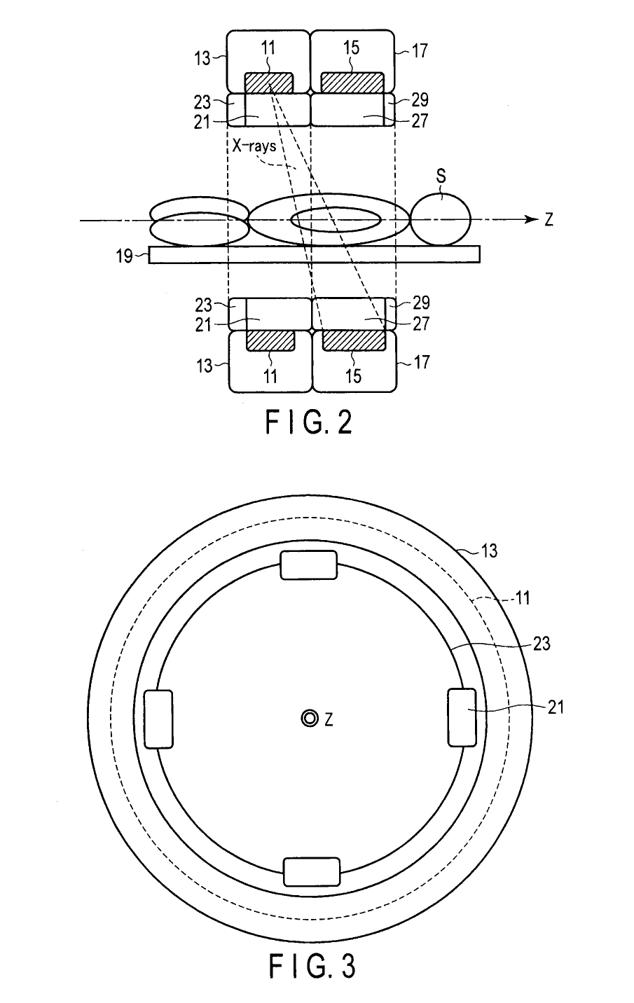

FIG. 2 is a view schematically showing the structure of the gantry 10. As shown in FIG. 2, the gantry 10 includes an annular structure (to be referred to as an X-ray source ring hereinafter) 13 accommodating a plurality of X-ray sources 11 and an annular structure (to be referred to as a detector ring hereinafter) 17 accommodating a plurality of X-ray detectors 15. The X-ray source ring 13 and the detector ring 17 are installed such that a central axis Z of the X-ray source ring 13 spatially coincides with that of the detector ring 17. The X-ray source ring 13 and the detector ring 17 are arranged side by side along the central axis Z. The X-ray source ring 13 and the detector ring 17 share an opening. The interior of the opening is set as an FOV (field of view). A top 19 supported on a bed (not shown) is inserted into the opening. A subject S is placed on the top 19. The top 19 is positioned such that an imaging region of the subject S is included in the FOV.

FIG. 3 is a schematic sectional view of the X-ray source ring 13. As shown in FIG. 3, the X-ray source ring 13 includes the plurality of X-ray sources 11 arrayed circumferentially. Each of the plurality of X-ray sources 11 generates X-rays. As the X-ray sources 11, cold cathode X-ray tubes are used. A vacuum is maintained in the interior of the X-ray source ring 13. That is, the X-ray source ring 13 functions as a vacuum vessel. This makes all the X-ray sources 11 be arranged in a vacuum. A plurality of wedge filters 21 are arranged outside the X-ray source ring 13 on its inner circumferential side. The plurality of wedge filters 21 are supported by, for example, an annular support member (to be referred to as a filter support member hereinafter) 23 so as to be rotatable about the central axis Z. Each wedge filter 21 is an X-ray attenuation filter for spatially unifying the dose of X-rays applied from each X-ray source 11 to the subject S. The number of wedge filters 21 to be installed is not specifically limited as long as it is one or more. More specifically, the number of wedge filters 21 to be installed is equal to the number of X-ray sources 11 simultaneously driven, i.e., the number of directions in which X-ray irradiation is simultaneously performed. In the case shown in FIG. 3, the number of wedge filters 21 installed is four. The filter support member 23 is connected to a filter driver 25. The filter driver 25 generates motive power under the control of a filter drive controller 63 included in the console 50. Upon receiving the motive power, the filter support member 23 rotates about the rotation axis Z at a constant angular velocity. The filter support member 23 rotates independently of the X-ray source ring 13. That is, the X-ray source ring 13 remains stationary even when the filter support member 23 rotates.

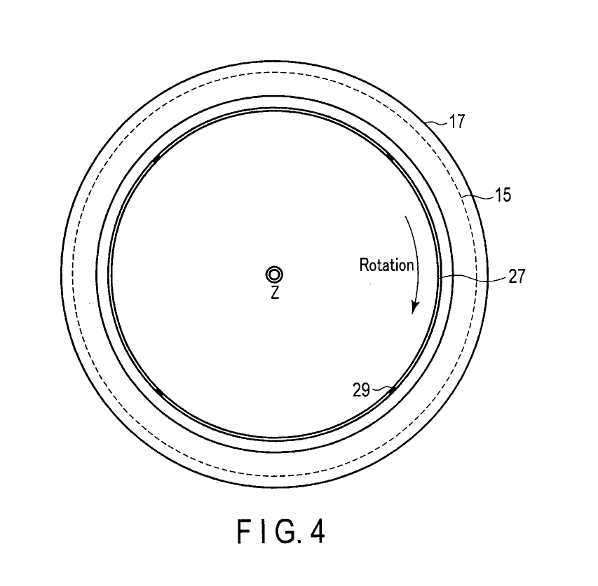

FIG. 4 is a schematic sectional view of the detector ring 17. As shown in FIG. 4, the detector ring 17 includes the plurality of X-ray detectors 15 arrayed on a circumference. Each X-ray detector 15 detects X-rays from the X-ray source ring 13 and generates an electrical signal corresponding to the intensity of detected X-rays. The X-ray detector 15 may be a direct detection type semiconductor detector or an indirect type detector constituted by a scintillator and a photodetector. A plurality of collimators (to be referred to as post-collimators hereinafter) 27 are arranged outside the detector ring 17 on its inner circumferential side. Each post-collimator 27 is a structure made of an X-ray attenuation material for limiting the solid angle of incident X-rays on the X-ray detector 15. It is preferable to provide, as each post-collimator 27, a collimator having the same structure as that used in the current third-generation CT. The plurality of post-collimators 27 are supported by, for example, an annular support member (to be referred to as a collimator support member hereinafter) 29 so as to be rotatable about the central axis Z. The number of post-collimators 27 to be installed is not specifically limited as long as it is one or more. Typically, the number of post-collimators 27 to be installed is equal to the number of wedge filters 21, i.e., the number of X-ray sources 11 simultaneous driven (that is, the number of directions in which X-ray irradiation is simultaneously performed). In the case shown in FIG. 4, the number of post-collimators 27 installed is four. The collimator support member 29 is connected to a collimator driver 31. The collimator driver 31 generates motive power under the control of a collimator drive controller 65 included in the console 50. Upon receiving the motive power, the collimator support member 29 rotates about the rotation axis Z at a constant angular velocity. The collimator support member 29 rotates independently of the detector ring 17. That is, the detector ring 17 remains stationary even when the collimator support member 29 rotates.

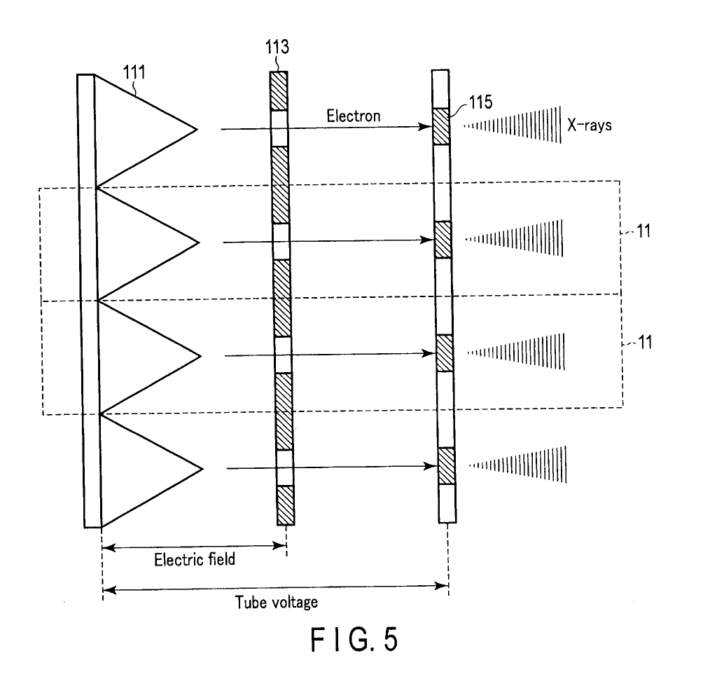

FIG. 5 is a view schematically showing the structure of each X-ray source 11. As shown in FIG. 5, the plurality of X-ray sources 11 are mounted on the X-ray source ring 13. Each X-ray source 11 includes a cold cathode electron source 111, a gate electrode 113, and an anode 115. The cold cathode electron source 111 is a material that emits electrons by using the field emission phenomenon. The field emission phenomenon is a phenomenon in which electrons in a metal placed in a high electric field exceed the work function and are emitted to the outside. A silicon or carbon nanotube is suitable as a material (to be referred to as an electric field emission material hereinafter) used for the cold cathode electron source 111. An electric field emission material is processed to have sharp tips, thereby forming a plurality of cold cathode electron sources 111. The plurality of cold cathode electron sources 111 are mounted on, for example, a semiconductor substrate. The plurality of cold cathode electron sources 111 are arranged to encircle the central axis Z inside the X-ray source ring 13.

As shown in FIG. 5, the plurality of gate electrodes 113 are arranged in front of the plurality of cold cathode electron sources 111. Each gate electrode 113 is an electrode for generating an electric field between itself and the cold cathode electron source 111. A gate driving circuit 33 is connected to each gate electrode 113. Each gate driving circuit 33 applies a gate pulse to the gate electrode 113 under the control of a gate controller 59 included in the console 50. Upon receiving the gate pulse, the gate electrode 113 generates an electric field between itself and the cold cathode electron source 111. The cold cathode electron source 111 in an electric field emits an electron from its tip according to the electric field emission phenomenon. The plurality of gate electrodes 113 are mounted on a semiconductor substrate. The plurality of gate electrodes 113 are arranged to encircle the central axis Z inside the X-ray source ring 13.

As shown in FIG. 5, the anodes 115 are arranged at positions to face the cold cathode electron sources 111 through the gate electrodes 113. For example, the anodes 115 are arranged to squarely face the cold cathode electron sources 111. The plurality of anodes 115 are mounted on a metal plate. The plurality of anodes 115 are arranged to encircle the central axis Z inside the X-ray source ring 13. Upon receiving electrons from the cold cathode electron sources 111, the anodes 115 generate X-rays. The anodes 115 and the cold cathode electron sources 111 are connected to a high voltage generator 35. The high voltage generator 35 applies voltages between the anodes 115 and the cold cathode electron sources 111 under the control of an X-ray controller 61 included in the console 50. The electrons emitted from the cold cathode electron sources 111 fly to the anodes 115 upon reception of the tube voltages and collide with the anodes 115. The collision of electrons with the anodes 115 generates X-rays. The generated X-rays are applied on the opposite side of the cold cathode electron sources 111 through the gate electrodes 113. The X-rays emitted from the X-ray sources 11 fly to the X-ray detectors 15 located on the opposite side of the rotation axis Z to the X-ray sources 11. The X-ray detectors 15 then detect the X-rays. In other words, the cold cathode electron sources 111 and the anodes 115 are positioned such that generated X-rays travel to the X-ray detectors 15 located on the opposite side to the X-ray sources 11.

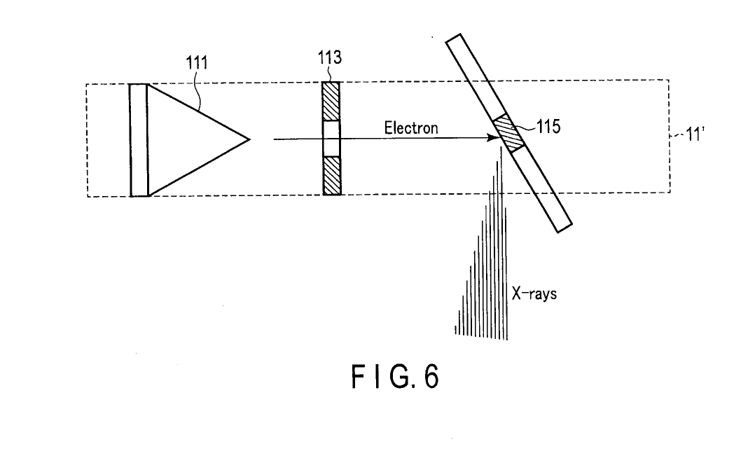

Note that the arrangement of each X-ray source 11 in FIG. 5 is merely an example. For example, referring to FIG. 5, each anode 115 is arranged to squarely face an electron stream, i.e., is of a target transmission type. However, this embodiment is not limited to this. For example, as shown in FIG. 6, the anode 115 may be tilted with respect to an electron stream, i.e., may be of a target reflection type. Even in this case, the cold cathode electron source 111 and the anode 115 are positioned such that generated X-rays travel to the X-ray detector 15 located on the opposite side to the X-ray source 11.

In addition, referring to FIG. 5, each X-ray source 11 includes one each of the cold cathode electron source 111, the gate electrode 113, and the anode 115. However, this embodiment is not limited to this. It is possible to individually increase or decrease the numbers of cold cathode electron sources 111, gate electrodes 113, and anodes 115 of each X-ray source 11. For example, one anode 115 may be provided for a plurality of cold cathode electron sources 111 or a plurality of anodes 115 may be provided for one cold cathode electron source 111.

In addition, it is preferable to provide the plurality of X-ray sources 11 on the X-ray source ring 13 along the Z-axis and provide the plurality of X-ray detectors 15 on the detector ring 17 along the Z-axis. This makes it possible to apply X-rays into a three-dimensional spatial region. Eventually, it is possible to perform volume scanning.

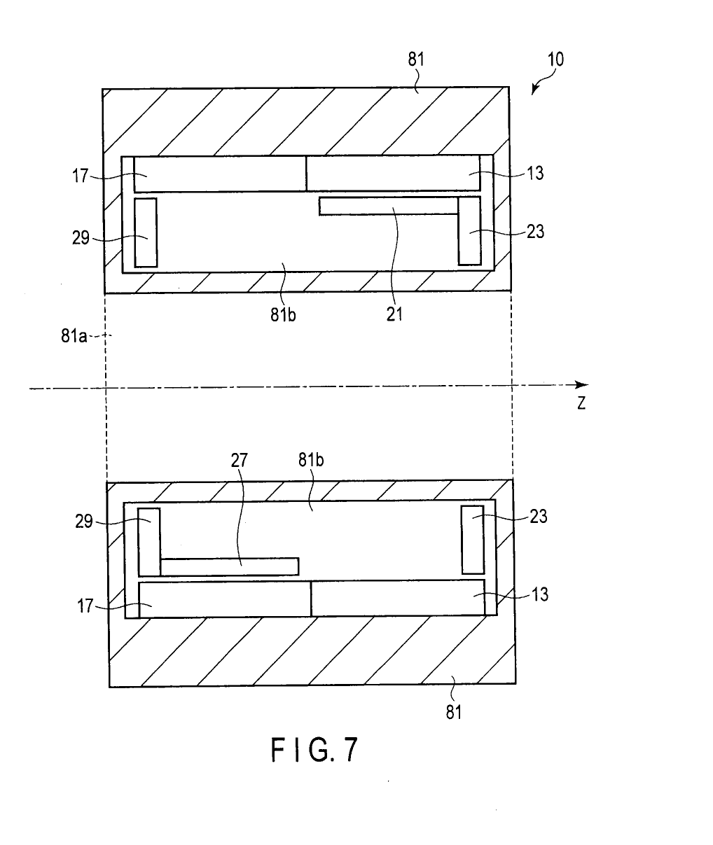

The typical structure of the gantry 10 according to this embodiment will be described in more detail below. FIG. 7 is a longitudinal sectional view of the gantry 10 according to the embodiment. As shown in FIG. 7, the gantry 10 includes a housing 81 having an opening 81a. The X-ray source ring 13 and the detector ring 17 are arrayed in an internal space 81b of the housing 81 along the rotation axis Z. The filter support member 23 which supports at least one wedge filter 21 is arranged on the inner circumferential side of the X-ray source ring 13. The filter support member 23 has an opening larger in diameter than the opening 81a and is arranged in the internal space 81b such that the central axis of the filter support member 23 coincides with the rotation axis Z. The collimator support member 29 which supports at least one post-collimator 27 is arranged on the inner circumferential side of the detector ring 17. The collimator support member 29 has an opening larger in diameter than the opening 81a and is arranged in the internal space 81b such that the central axis of the collimator support member 29 coincides with the rotation axis Z. The filter driver 25 and the collimator driver 31 (neither of which is shown in FIG. 7) respectively rotate the filter support member 23 and the collimator support member 29 about the rotation axis Z.

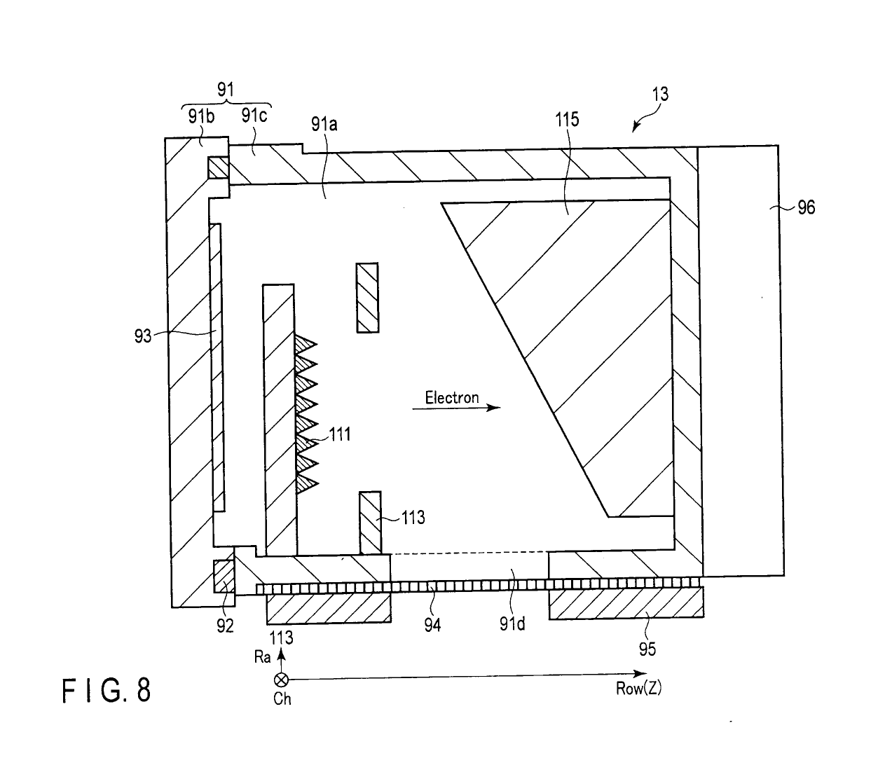

FIG. 8 is a longitudinal sectional view showing the detailed structure of the X-ray source ring 13. Note that a direction along the rotation axis Z of the X-ray source ring 13 will be referred to as a row direction (Row direction), and the circumferential direction of the X-ray source ring 13 will be referred to as a channel direction (Ch direction). A direction orthogonal to the row direction and channel direction coincides with the radial direction (Ra direction) of the X-ray source ring 13. As shown in FIG. 8, the X-ray source ring 13 includes a housing 91 having an annular shape whose central axis coincides with the rotation axis Z. The housing 91 has a hollow structure. A vacuum is maintained in an internal space 91a of the housing 91. More specifically, the housing 91 includes a lid 91b and a vessel 91c each having an annular shape whose central axis coincides with the rotation axis Z. The lid 91b and the vessel 91c are preferably formed from a robust material such as iron or stainless steel. The lid 91b and the vessel 91c are preferably fastened to each other with a fastening tool or the like for accurately keeping a vacuum in the internal space 91a. For example, the lid 91b and the vessel 91c are fastened to each other through a gasket 92. As the gasket 92 according to this embodiment, it is possible to use any of the existing types of gaskets such as a nonmetallic gasket, a semi-metallic gasket, and a metallic gasket. The inner surface of the lid 91b is provided with a getter 93 which adsorbs a residual gas in the internal space 91a. As the getter 93 according to this embodiment, it is possible to use either a contact getter or a diffusion getter. As the getter 93, for example, it is possible to use any of the existing metals such as titanium and a barium-aluminum alloy.

The plurality of cold cathode electron sources 111 are provided on the X-ray detection ring 17 side of the X-ray source ring 13. The plurality of cold cathode electron sources 111 are arrayed along the channel direction and the radial direction. For example, the plurality of cold cathode electron sources 111 are fixed to a support member 111a. The support member 111a is fixed to the inner surface of the vessel 91c. The anode 115 is provided on the opposite side to the plurality of cold cathode electron sources 111 in the row direction. The plurality of anodes 115 may be arrayed in the internal space 91a of the housing 91 along the channel direction or the anode 115 having an annular shape whose central axis coincides with the rotation axis Z may be provided. To allow the anode 115 to irradiate the X-ray detection ring 17, which is adjacent to it along the rotation axis Z, with X-rays, the anode 115 is sloped so as to gradually decrease in thickness in the row direction toward the rotation axis Z along the radial direction. The gate electrodes 113 are provided between the anode 115 and the plurality of cold cathode electron sources 111 in the row direction. The plurality of gate electrodes 113 are arrayed along the channel direction. When, for example, applying X-rays from 1,000 directions around the rotation axis Z, it is preferable to provide 1,000 gate electrodes 113 around the rotation axis Z. One gate electrode 113 is provided for a predetermined number of cold cathode electron sources 111 adjacent to each other in the channel direction. The predetermined number may be any number equal to or more than one. The gate electrodes 113 are fixed to, for example, the inner surface of the vessel 91c.

An exit port 91d for X-rays generated from the anode 115 is formed in the vessel 91c. The exit port 91d is formed in the vessel 91c so as to encircle the rotation axis Z. An X-ray filter 94 is attached to the outer wall of the vessel 91c so as to cover the exit port 91d. The X-ray filter 94 absorbs small-dose components of X-rays passing through the exit port 91d. The outer wall of the vessel 91c is provided with a slit 95 through the X-ray filter 94. The slit 95 limits the irradiation field of X-rays. Note that the slit 95 may be provided so as to be rotatable about the rotation axis Z in synchronism with the wedge filters 21.

The outer wall of the vessel 91c is provided with a cooling unit 96 which cools the X-ray source ring 13. As the cooling unit 96, it is possible to use any apparatus, tool, or material which can cool the X-ray source ring 13. For example, a cooling pipe through which a refrigerant passes can be used as the cooling unit 96. The main heat source of the X-ray source ring 13 is the anode 115 which generates heat upon receiving electrons from the cold cathode electron source 111. Therefore, the cooling unit 96 is preferably provided on the opposite side of the vessel 91c to the anode 115 to efficiently cool the anode 115.

As shown in FIG. 1, a data collection circuit 37 is connected to the plurality of X-ray detectors 15. The data collection circuit 37 reads out electrical signals generated by the plurality of X-ray detectors 15 under the control of an imaging controller 67, and converts the readout electrical signals into digital data by A/D conversion. More specifically, the data collection circuit 37 reads out electrical signals from the X-ray detectors 15 for each view and converts them into digital data. Digital data after conversion will be referred to as raw data. Raw data is supplied to the console 50. Note that a view corresponds to a sampling period for raw data from each X-ray detector 15, in other words, a duration of X-ray irradiation from each X-ray source 11.

As shown in FIG. 1, the console 50 includes a system controller 51 as a main unit, a preprocessor 53, a reconstruction unit 55, an image processing unit 57, the gate controller 59, the X-ray controller 61, the filter drive controller 63, a collimator drive controller 65, the imaging controller 67, a display 69, an input unit 71, and a storage 73.

The preprocessor 53 preprocesses raw data from the data collection circuit 37. As preprocessing, the same processing as that used in the third-generation CT is used. More specifically, preprocessing includes logarithmic conversion, X-ray intensity correction, and offset correction.

The reconstruction unit 55 generates a CT image expressing the spatial distribution of CT values by applying an image reconstruction algorithm for preprocessed raw data. As an image reconstruction algorithm, there may be used any of the existing image reconstruction algorithms including analytical image reconstruction methods such as the FBP (filtered back projection) method and the CBP (convolution back projection) method and statistical image reconstruction methods such as the ML-EM (maximum likelihood expectation maximization) method and the OS-EM (ordered subset expectation maximization) method.

The image processing unit 57 performs various types of image processing for a CT image. For example, the image processing unit 57 performs volume rendering, surface rendering, pixel value projection processing, pixel value conversion, and the like.

The gate controller 59 controls the plurality of gate driving circuits 33 to cause the plurality of X-ray sources 11 to generate X-rays in accordance with a preset order under the control of the imaging controller 67. More specifically, the gate controller 59 supplies a timing pulse to the gate driving circuit 33 connected to the X-ray source 11 as an X-ray generation target. Upon receiving the timing pulse, the gate driving circuit 33 immediately applies a gate pulse to the gate electrode 113 for the X-ray source 11 as the connection destination. Upon application of the gate pulse, as described above, the cold cathode electron source 111 emits electrons according to the electric field emission phenomenon. The electrons then collide with the anode 115 to generate X-rays. The order of generation of X-rays from the X-ray sources 11 (switching of the X-ray source 11 as an X-ray generation target) will be briefly described below. The X-ray source 11 as an X-ray generation target is switched among the plurality of X-ray sources 11 accommodated in the X-ray source ring 13 in accordance with a preset order for each view. The X-ray source 11 as an X-ray generation target is sequentially switched among the X-ray sources 11 along a circumference for each view. In this case, the gate controller 59 controls the plurality of gate driving circuits 33 so as to cause the plurality of X-ray sources 11 to sequentially generate X-rays around the circumference of the X-ray source ring 13. In other words, the gate controller 59 controls the plurality of gate driving circuits 33 so as to cause the plurality of cold cathode electron sources 111 to sequentially generate electrons around the circumference of the X-ray source ring 13. In this case, the gate driving circuits 33 may be driven to generate X-rays from one X-ray source 11 for each view or to generate X-rays from the plurality of X-ray sources 11 for each view. For example, it is preferable to drive the plurality of gate driving circuits 33 to simultaneously generate X-rays from the four X-ray sources 11 separated from each other at equal intervals for each view.

The X-ray controller 61 controls the high voltage generator 35 to apply a tube voltage corresponding to a predetermined X-ray condition between the cold cathode electron source 111 and the anode 115 under the control of the imaging controller 67. More specifically, the X-ray controller 61 supplies a timing pulse to the high voltage generator 35 to apply a tube voltage to the X-ray source 11 as an X-ray generation target in synchronism with the application timing of a gate pulse to the gate electrode 113 included in the X-ray source 11. Upon receiving the timing pulse, the high voltage generator 35 immediately applies a tube voltage between the cold cathode electron source 111 and the anode 115 of the X-ray source 11 as the X-ray generation target. The electrons generated from the cold cathode electron source 111 upon application of the tube voltage collide with the anode 115 to generate X-rays. Note that a tube voltage application target is not limited to the X-ray source 11 as an X-ray generation target. That is, a tube voltage may be applied to the X-ray source 11 from which no X-rays are generated.

The filter drive controller 63 controls the filter driver 25 to rotate the plurality of wedge filters 21 around the rotation axis Z under the control of the imaging controller 67. More specifically, the filter drive controller 63 supplies a driving pulse to the filter driver 25 in synchronism with the application timing of a gate pulse to the gate electrode 113 of the X-ray source 11 as an X-ray generation target, in other words, in synchronism with the generation of X-rays from the X-ray source 11. Upon receiving the driving pulse, the filter driver 25 drives the filter support member 23 to rotate the plurality of wedge filters 21 around the rotation axis Z at an angular velocity corresponding to the pulse interval between driving pulses. More specifically, the filter support member 23 is rotated to always position the wedge filter 21 in front of the X-ray source 11 as an X-ray generation target, which is switched for each view, regardless of switching of the X-ray 11. In other words, the filter support member 23 is rotated to position the wedge filter 21 in front of the X-ray generation portion of the X-ray source ring 13. The filter support member 23 may be continuously rotated or may be intermittently rotated to stop when X-rays are generated.

The collimator drive controller 65 controls the collimator driver 31 to rotate the plurality of post-collimators 27 around the rotation axis Z under the control of the imaging controller 67. More specifically, the collimator drive controller 65 supplies a driving pulse to the collimator driver 31 in synchronism with the application timing of a gate pulse to the gate electrode 113 of the X-ray source 11 as an X-ray generation target, in other words, in synchronism with the generation of X-rays from the X-ray source 11. Upon receiving the driving pulse, the collimator driver 31 drives the collimator support member 29 to rotate the plurality of post-collimators 27 around the rotation axis Z at an angular velocity corresponding to the pulse interval between driving pulses. More specifically, the collimator support member 29 is rotated to always position the post-collimator 27 in front of the X-ray detector 15 located on the opposite side of the rotation axis Z to the X-ray source 11 as an X-ray generation target, which is switched for each view, regardless of switching of the X-ray source 11. In other words, the collimator support member 29 is rotated to position the post-collimator 27 in front of the X-ray detector 15 located on the opposite side of the rotation axis Z to the X-ray generation portion of the X-ray source ring 13. The collimator support member 29 may be continuously rotated or may be intermittently rotated to stop when X-rays are generated.

The imaging controller 67 synchronously controls the gate controller 59, the X-ray controller 61, the filter drive controller 63, the collimator drive controller 65, and the data collection circuit 37. More specifically, the imaging controller 67 synchronously outputs commands to the gate controller 59 and the X-ray controller 61 to switch the X-ray source 11 as an X-ray generation target in synchronism with the switching timing of a view. In addition, the imaging controller 67 synchronously outputs commands to the filter drive controller 63 and the collimator drive controller 65 to place the wedge filter 21 in front of the X-ray source 11 as an X-ray generation target and to place the post-collimator 27 in front of the X-ray detector 15 located on the opposite side of the rotation axis Z to the X-ray source 11. In other words, the imaging controller 67 synchronously outputs commands to the filter drive controller 63 and the collimator drive controller 65 to position the wedge filter 21 in front of the X-ray generation portion of the X-ray source ring 13 and position the post-collimator 27 in front of the X-ray detector 15 located on the opposite side of the rotation axis Z to the X-ray generation portion. In addition, the imaging controller 67 controls the data collection circuit 37 to read out electrical signals from the X-ray detector 15 in synchronism with the switching timing of a view. The switching timing of a view may be defined by the timing at which the filter support member 23 or the collimator support member 29 generates a trigger signal every time the filter support member 23 or the collimator support member 29 rotates through a predetermined angle or may be defined by the generation timing of a frequency division signal of a clock signal from a clock circuit of the imaging controller 67 (or the system controller 51).

The display 69 displays various types of information. For example, the display 69 displays the CT image generated by the reconstruction unit 55, a CT image after image processing by the image processing unit 57, or the like. In addition, the display 69 displays a setting screen for imaging conditions or the like. As a display 69, it is possible to use, for example, a CRT display, liquid crystal display, organic EL display, or plasma display.

The input unit 71 accepts various types of commands or information inputs from the user of an input device. As an input device, it is possible to use a keyboard, a mouse, various types of switches, and the like.

The storage 73 is a storage device which stores various types of information. For example, the storage 73 stores raw data and CT images. In addition, the storage 73 stores an imaging program according to this embodiment.

The system controller 51 functions as the main unit of the X-ray computed tomography apparatus. The system controller 51 reads out an imaging program according to this embodiment from the storage, and controls various types of constituent elements in accordance with the imaging program, thereby performing imaging processing according to the embodiment.

An operation example in imaging processing performed by the X-ray computed tomography apparatus under the control of the system controller 51 will be described next.

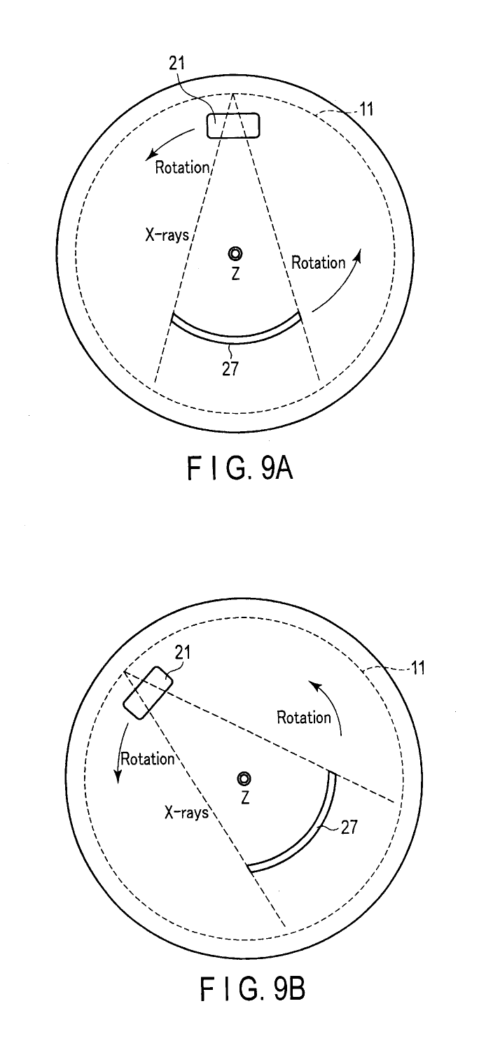

FIGS. 9A and 9B are plan views showing the placement of the X-ray source 11, the wedge filter 21, and the post-collimator 27 when the number of X-ray sources simultaneously driven is one. FIG. 9A shows the placement at time t. FIG. 9B shows the placement at time t+Δt. The imaging controller 67 synchronously controls the gate controller 59, the X-ray controller 61, the filter drive controller 63, the collimator drive controller 65, and the data collection circuit 37 so as to sequentially switch the X-ray source 11 as an X-ray generation target around the rotation axis Z, place the wedge filter 21 in front of the X-ray source 11 as the X-ray generation target, and place the post-collimator 27 in front of the X-ray detector 15 facing the X-ray source 11 as the X-ray generation target. In this case, the plurality of X-ray sources 11 and the plurality of X-ray detectors 15 are fixed without being rotated.

More specifically, in an imaging period, the X-ray source 11 as an X-ray generation target is sequentially switched along a circumference for each set of a predetermined number of views so as to apply X-rays from the entire angle range necessary for image reconstruction. When, for example, 360° reconstruction is to be performed, an X-ray source as an X-ray generation target is sequentially and electrically switched along the circumference for each set of a predetermined number of views so as to apply X-rays from all directions in an imaging period. The predetermined number of views can be set to an arbitrary number equal to or more than one. The wedge filter 21 and the post-collimator 27 rotate in synchronism with switching of the X-ray source 11 so as to place the wedge filter 21 in front of the X-ray source 11 as an X-ray generation target and place the post-collimator 27 in front of the X-ray detector 15 facing the X-ray source 11 over an imaging period.

The data collection circuit 37 collects the electrical signals generated by the X-ray detectors 15. For example, the data collection circuit 37 collects data (to be referred to as an intensity value record hereinafter) representing a digital value (to be referred to as an intensity value hereinafter) corresponding to the intensity of X-rays for each address (a combination of a channel and a row) of the X-ray detector which has detected the X-rays. The data collection circuit 37 generates a set of intensity value records concerning all addresses associated with the same imaging angle as raw data. In this case, an imaging angle is defined as the angle, around the rotation axis Z, of the X-ray source 11 which has applied detected X-rays. When raw data in an angle range necessary for image reconstruction is collected in this manner, the imaging controller 67 terminates the imaging operation. The preprocessor 53 then performs preprocessing for the raw data. Then reconstruction unit 55 generates a CT image based on the raw data after the preprocessing. The display 69 displays the generated CT image.

Even the X-ray computed tomography apparatus including the X-ray source ring 13 and the detector ring 17 can perform CT imaging similar to that in third-generation CT by moving the X-ray generation portion along a circumference by electrically switching the gate electrodes 113 upon fixing the spatial positions of the plurality of X-ray sources 11 arrayed on the circumference. The gate controller 59 switches the gate electrodes 113 at high speed. The X-ray computed tomography apparatus according to this embodiment can therefore shorten the imaging time as compared with the third-generation CT designed to rotate a heavy rotating ring as in the related art. In addition, as in third-generation CT, the X-ray computed tomography apparatus according to the embodiment can suppress the exposure dose of the subject S and reduce the amount of scattered radiation detected by rotating the wedge filter 21 and the post-collimator 27 in synchronism with switching of the X-ray source 11. Note that the filter support member 23 equipped with the wedge filters 21 and the collimator support member 29 equipped with the post-collimators 27 are lighter in weight than the rotating ring in the third-generation CT, which is equipped with an X-ray tube, a high voltage generator, an X-ray detector, and the like. The centrifugal force accompanying the rotation of the filter support member 23 and the collimator support member 29 is lower than that accompanying the rotation of the rotating ring in the third-generation CT. The X-ray computed tomography apparatus according to this embodiment can therefore rotate the filter support member 23 and the collimator support member 29 at a high speed corresponding to the switching speed of the gate electrode 113.

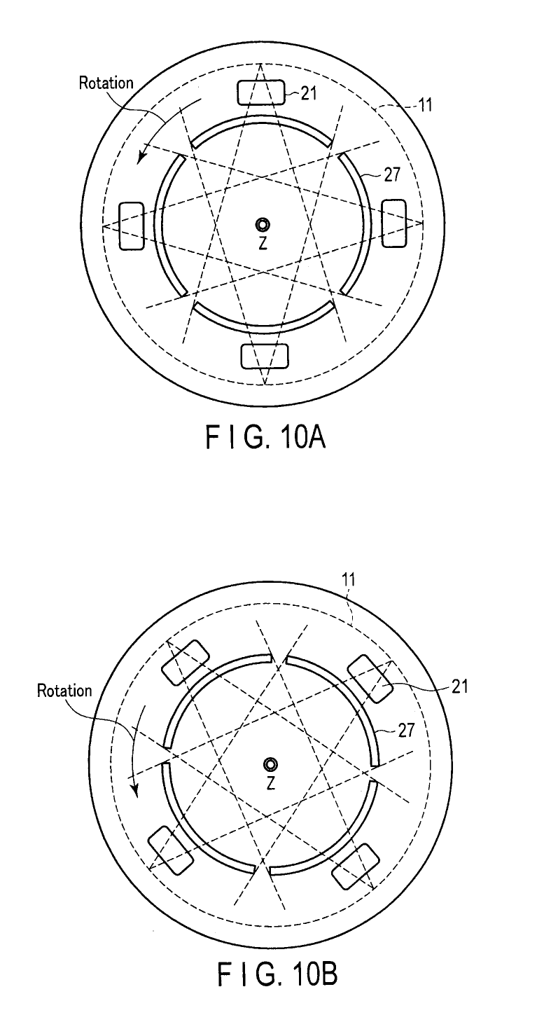

An operation example in imaging processing when the number of X-ray sources simultaneously driven is four will be described next. FIGS. 10A and 10B are plan views each showing the placement of the X-ray sources 11, the wedge filters 21, and the post-collimators 27 when the number of X-ray sources simultaneously driven is four. FIG. 10A shows the placement at time t. FIG. 10B shows the placement at time t+Δt. A combination of the X-ray source 11, the wedge filter 21, and the post-collimator 27 serves as one X-ray irradiation system in CT. Simultaneously driving four X-ray sources is synonymous with having four X-ray irradiation systems. Referring to FIGS. 10A and 10B, the four X-ray sources 11 as X-ray generation targets are set to be separated from each other at 90° intervals for each view. The imaging controller 67 synchronously controls the gate controller 59, the X-ray controller 61, the filter drive controller 63, the collimator drive controller 65, and the data collection circuit 37 to sequentially switch the four X-ray sources 11 as X-ray generation targets along the circumference, respectively arrange the wedge filters 21 in front of the four X-ray sources 11, and respectively arrange the post-collimators 27 in front of the X-ray detectors 15 each located on the opposite side of the rotation axis Z to a corresponding one of the X-ray sources 11. In this case, the plurality of X-ray sources 11 and the plurality of X-ray detectors 15 are fixed without being rotated.

More specifically, the X-ray sources 11 as X-ray generation targets are sequentially switched along the circumference for each set of a predetermined number of views so as to apply X-rays from all the angle ranges necessary for image reconstruction. When, for example, 360° reconstruction is to be performed, the X-ray sources 11 as X-ray generation targets are sequentially switched along the circumference for each set of a predetermined number of views so as to apply X-rays from all directions in an imaging period. Note that the predetermined number of views can be set to an arbitrary number equal to or more than one. The four wedge filters 21 and the four post-collimators 27 are rotated in synchronism with switching of the X-ray sources 11 as X-ray generation targets so as to respectively arrange the four wedge filters 21 in front of the four X-ray sources 11 as the X-ray generation targets and respectively arrange the four post-collimators 27 in front of the four X-ray detectors 15 each located on the opposite side to a corresponding one of the four X-ray sources 11 as the X-ray generation targets over an imaging period.

When the number of X-ray sources simultaneously driven is four, it is possible to shorten the imaging time to ¼ that when the number of X-ray sources simultaneously driven is one, by using the same material for all the wedge filters 21 and all the post-collimators 27 and applying the same tube voltage to all the X-ray sources 11. In addition, when rotating the wedge filters 21 and the post-collimators 27 at the same rotational speed as that in the current third-generation CT, it is possible to shorten the imaging time to 70 ms or less. This makes it possible to execute cardiac CT with respect to even the subject S with a heartbeat of 100 or more without any medication. As described above, the X-ray computed tomography apparatus according to this embodiment can greatly reduce the weight of the rotating portion as compared with the third-generation CT, and hence can implement imaging at a high speed of 50 ms or less when rotating the wedge filters 21 and the post-collimators 27 with the same centrifugal force as that in the current third-generation CT.

The data collection circuit 37 collects the electrical signals generated by the X-ray detectors 15 as raw data. For example, the data collection circuit 37 collects an intensity value record representing a digital value (intensity value) corresponding to the intensity of X-rays for each of the addresses of the X-ray detectors 15 which have detected the X-rays. The data collection circuit 37 generates a set of intensity value records concerning all addresses associated with the same imaging angle as raw data. When raw data in an angle range necessary for image reconstruction is collected in this manner, the imaging controller 67 terminates the imaging operation. The preprocessor 53 then performs preprocessing for the raw data. Then reconstruction unit 55 generates a CT image based on the preprocessed raw data. The display 69 displays the generated CT image.

### Application Example

In the above embodiment, even if a plurality of X-ray sources are simultaneously driven, single-energy CT is executed. However, this embodiment is not limited to this. An X-ray computed tomography apparatus according to an application example of this embodiment can execute spectral CT (multi-energy CT) when a plurality of X-ray sources are simultaneously driven. The X-ray computed tomography apparatus according to this application example will be described below.

The X-ray computed tomography apparatus according to this embodiment can execute tube-voltage-based spectral CT and filter-based spectral CT. Tube-voltage-based spectral CT will be described first. Note that the X-ray computed tomography apparatus according to the embodiment can perform spectral CT without any limitation on the number of X-ray sources simultaneously driven. However, for the sake of a concrete description of the embodiment, assume that the number of X-ray sources simultaneously driven is three.

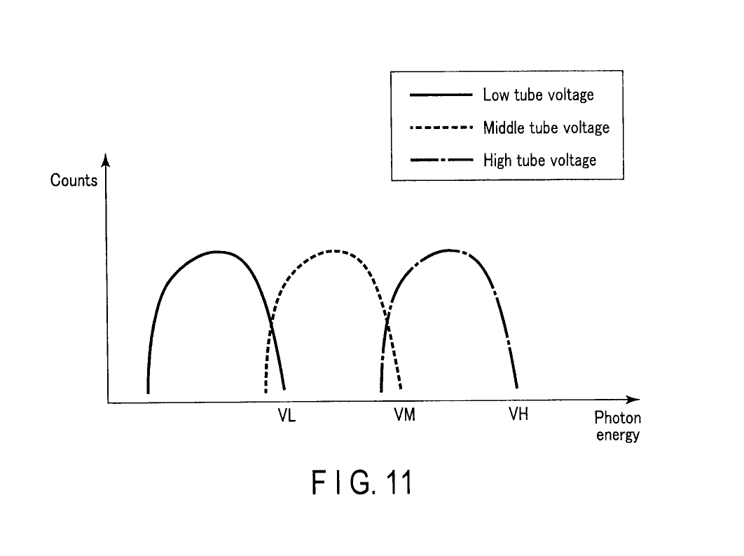

FIG. 11 is a graph schematically showing the energy spectra of X-rays generated from the X-ray source 11 upon reception of different tube voltages. The ordinate of FIG. 11 is defined as the counts of X-rays entering the X-ray detector 15. The abscissa of FIG. 11 is defined as photon energy. The solid line in FIG. 11 represents the energy spectrum of X-rays generated from the X-ray source 11 upon reception of a low tube voltage, and represents an energy distribution corresponding to the low tube voltage, with an energy value VL being the maximum. Likewise, the dotted line in FIG. 11 represents the energy spectrum of X-rays generated from the X-ray source 11 upon reception of a middle tube voltage, and represents an energy distribution corresponding to the middle tube voltage, with an energy value VM being the maximum. The one-dot dashed line in FIG. 11 represents the energy spectrum of X-rays generated from the X-ray source 11 upon reception of a high tube voltage, and represents an energy distribution corresponding to the high tube voltage, with an energy value VH being the maximum. Note that the low tube voltage, the middle tube voltage, and the high tube voltage increase in value in the order named. Discretely setting a plurality of tube voltage values for the plurality of X-ray sources 11 in this manner will separate the energy ranges of X-rays generated from the plurality of X-ray sources 11 from each other. This makes it possible to perform spectral CT.

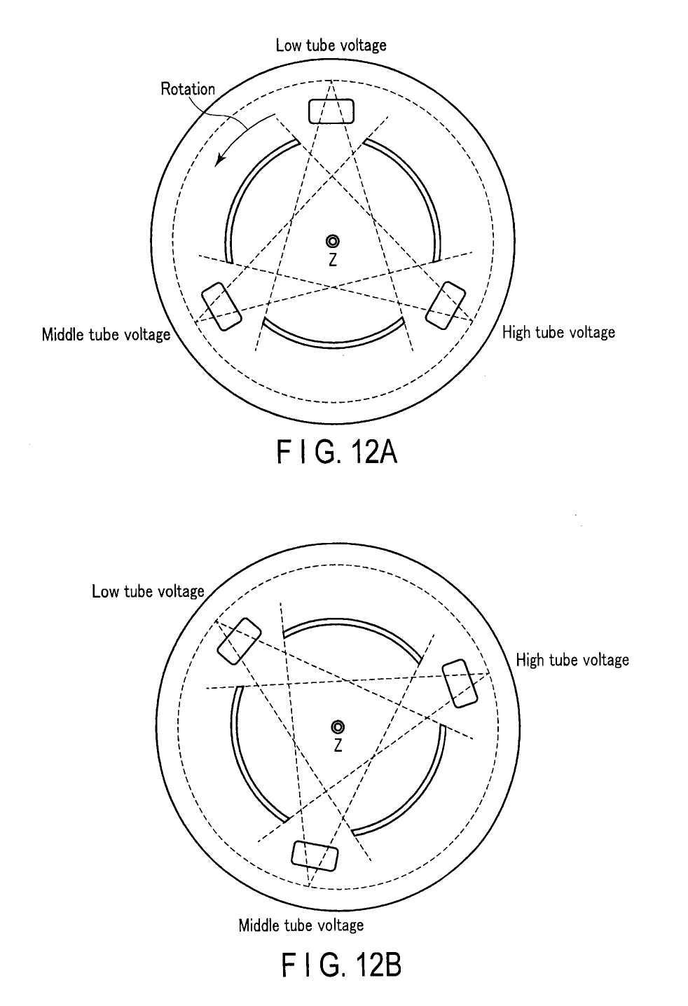

FIGS. 12A and 12B are plan views each showing the placement of the X-ray sources 11, the wedge filters 21, and the post-collimators 27 when the number of X-ray sources simultaneously driven is three in tube-voltage-based spectral CT. FIG. 12A shows the placement at time t. FIG. 12B shows the placement at time t+Δt. As described above, when the number of X-ray sources simultaneously driven is three, the three wedge filters 21 are held by the filter support member 23 along a circumference at equal intervals, and the three post-collimators 27 are held by the collimator support member 29. The three wedge filters 21 are formed from the same material to make them exhibit the same X-ray attenuation effect with respect to X-rays from the three X-ray sources 11.

When performing tube-voltage-based spectral CT, the imaging controller 67 synchronously controls the gate controller 59, the filter drive controller 63, the collimator drive controller 65, and the data collection circuit 37 to sequentially switch the three X-ray sources as X-ray generation targets along a circumference, arrange the wedge filter 21 in front of each of the three X-ray sources 11 as X-ray generation targets, and arrange the post-collimator 27 in front of each X-ray detector 15 located on the opposite side of the rotation axis Z to a corresponding one of the X-ray sources 11 as X-ray generation targets. In this case, the imaging controller 67 controls the gate controller 59 and the X-ray controller 61 to perform X-ray irradiation in the same angle range necessary for image reconstruction with each of three tube voltages. When, for example, 360° reconstruction is to be performed, X-ray irradiation is performed throughout 360° starting from each of different angles with three tube voltages. In the case shown in FIGS. 12A and 12B, irradiation of low-tube-voltage X-rays is performed in the angle range of 0° to 360°, irradiation of middle-tube-voltage X-rays is performed in the angle range of 120° to 480°, and irradiation of high-tube-voltage X-rays is performed in the angle range of 240° to 600°.

The data collection circuit 37 collects raw data from each X-ray detector 15 for each view. In this case, raw data originating from the X-rays generated from the X-ray source 11 upon application of a high tube voltage is called high-tube-voltage raw data, raw data originating from the X-rays generated from the X-ray source 11 upon application of a middle tube voltage is called middle-tube-voltage raw data, and raw data originating from the X-rays generated from the X-ray source 11 upon application of a low tube voltage is called low-tube-voltage raw data. The reconstruction unit 55 reconstructs a CT image (high-tube-voltage CT image) based on high-tube-voltage raw data, a CT image (middle-tube-voltage CT image) based on middle-tube-voltage raw data, and a CT image (low-tube-voltage CT image) based on low-tube-voltage raw data. In addition, the reconstruction unit 55 may generate an image concerning a base material (a base material image) based on high-tube-voltage raw data, middle-tube-voltage raw data, and low-tube-voltage raw data or may generate a monochromatic X-ray image, a density image, and an effective atomic number image, each based on the base material. The display 69 displays the high-tube-voltage CT image, middle-tube-voltage CT image, low-tube-voltage CT image, base material image, monochromatic X-ray image, density image, and effective atomic number image.

With the above arrangement, the X-ray computed tomography apparatus including the X-ray source ring 13 and the detector ring 17 implements tube-voltage-based spectral CT.

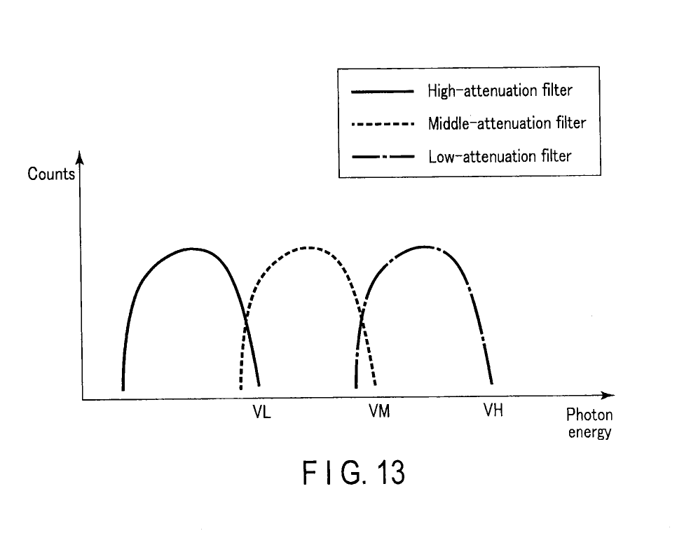

Filter-based spectral CT will be described next. FIG. 13 is a graph schematically showing the energy spectra of X-rays generated from the X-ray sources 11 and transmitted through the wedge filters 21 with different X-ray attenuation coefficients. The ordinate of FIG. 13 is defined as the counts of X-rays entering the X-ray detector 15. The abscissa of FIG. 13 is defined as photon energy. The solid line in FIG. 13 represents the energy spectrum of X-rays transmitted through the wedge filter 21 with a low X-ray attenuation coefficient, and represents an energy distribution with the energy value VL being the maximum. Likewise, the dotted line in FIG. 13 represents the energy spectrum of X-rays transmitted through the wedge filter 21 with a middle X-ray attenuation coefficient, and represents an energy distribution with the energy value VM being the maximum. The one-dot dashed line in FIG. 13 represents the energy spectrum of X-rays transmitted through the wedge filter 21 with a low X-ray attenuation coefficient, and represents an energy distribution with the energy value VH being the maximum. Discretely setting a plurality of X-ray attenuation coefficients for the plurality of wedge filters 21 in this manner will separate the energy ranges of X-rays transmitted through the plurality of wedge filters 21 from each other. This makes it possible to perform spectral CT.

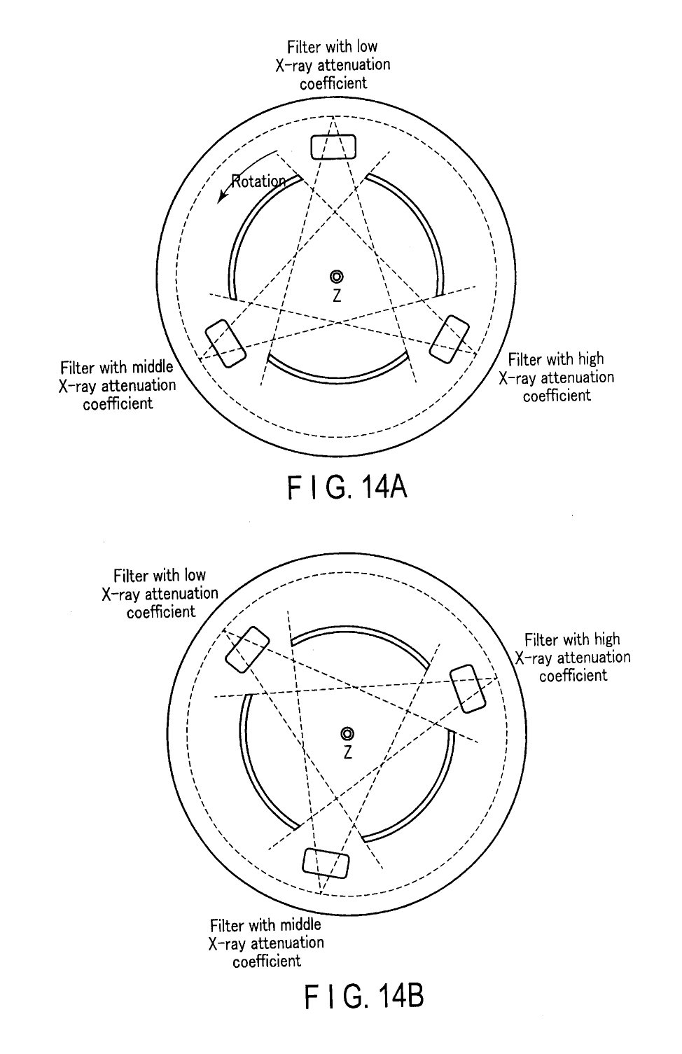

FIGS. 14A and 14B are plan views each showing the placement of the X-ray sources 11, the wedge filters 21, and the post-collimators when the number of X-ray sources simultaneously driven is three in filter-based spectral CT. FIG. 14A shows the placement at time t. FIG. 14B shows the placement at time t+Δt. As described above, when the number of X-ray sources simultaneously driven is three, the three wedge filters 21 are held by the filter support member 23 along a circumference at equal intervals, and the three post-collimators 27 are held by the collimator support member 29. The three wedge filters 21 are formed from different materials to make them exhibit different X-ray attenuation effects with respect to X-rays from the three X-ray sources 11. For example, the respective wedge filters 21 are preferably formed from arbitrary metals with different X-ray attenuation coefficients. More specifically, the first, second, and third wedge filters are preferably formed from copper, iodine, and gadolinium, respectively.

As in tube-voltage-based spectral CT, the data collection circuit 37 collects raw data from the respective X-ray detectors 15 for each view. In this case, raw data originating from the X-rays transmitted through the wedge filter 21 with a low X-ray attenuation coefficient is called high-energy raw data, raw data originating from the X-rays transmitted through the wedge filter 21 with a middle X-ray attenuation coefficient is called middle-energy raw data, and raw data originating from the X-rays transmitted through the wedge filter 21 with a high X-ray attenuation coefficient is called low-energy raw data. The reconstruction unit 55 reconstructs a CT image (high-energy CT image) based on the high-energy raw data, a CT image (middle-energy CT image) based on the middle-energy raw data, and a CT image (low-energy CT image) based on the low-energy raw data. A high-energy CT image is substantially equivalent to a high-tube-voltage CT image. A middle-energy CT image is substantially equivalent to a middle-tube-voltage CT image. A low-energy CT image is substantially equivalent to a low-tube-voltage CT image. In addition, the reconstruction unit 55 may generate an image concerning a predetermined base material (a base material image) based on high-energy raw data, middle-energy raw data, and low-energy raw data or may generate a monochromatic X-ray image, a density image, and an effective atomic number image, each based on the base material. The display 69 displays the high-energy CT image, middle-energy CT image, low-energy CT image, base material image, monochromatic X-ray image, density image, and effective atomic number image.

With the above arrangement, the X-ray computed tomography apparatus including the X-ray source ring 13 and the detector ring 17 implements filter-based spectral CT.

Note that in the above description, spectral CT is executed by individually adjusting tube voltages and materials for the wedge filters. However, this embodiment is not limited to this. That is, spectral CT may be executed by optimizing both tube voltages and materials for the wedge filters. In this case, it is preferable to adjust both tube voltages and materials for the wedge filters so as to separate the energy range of X-rays from each X-ray irradiation system constituted by one X-ray source 11, one wedge filter 21, and one post-collimator 27 from the energy range of X-rays from another X-ray irradiation system.

The first embodiment can therefore provide an X-ray computed tomography apparatus which can execute high-speed imaging.

### Second Embodiment

A photon counting CT apparatus according to the second embodiment will be described next. Note that in the following description, the same reference numerals denote constituent elements having almost the same functions as those in the first embodiment, and a repetitive description will be made only when required.

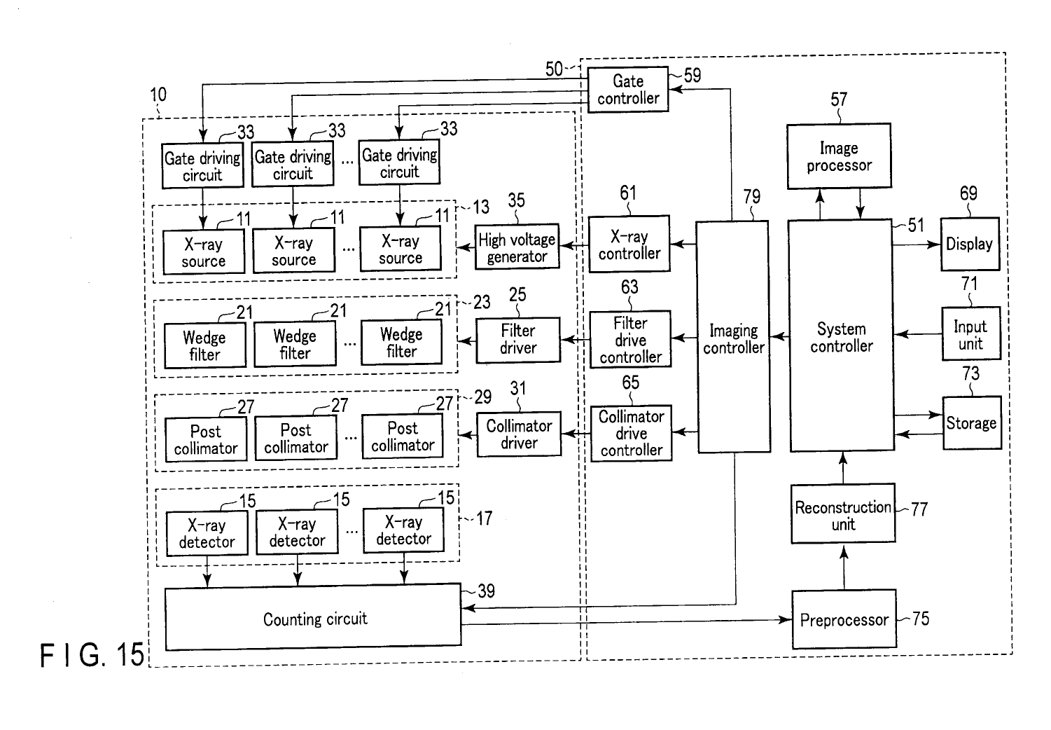

FIG. 15 shows the arrangement of the photon counting CT apparatus according to the second embodiment. As shown in FIG. 15, the photon counting CT apparatus according to the second embodiment includes a counting circuit 39 in place of the data collection circuit 37 of the X-ray computed tomography apparatus according to the first embodiment, a preprocessor 75 in place of the preprocessor 53, a reconstruction unit 77 in place of the reconstruction unit 55, and an imaging controller 79 in place of the imaging controller 67.

The counting circuit 39 counts the numbers of counts of X-ray photons detected by X-ray detectors 15 in a plurality of energy bands under the control of the imaging controller 79. As counting schemes used by the counting circuit 39, the sinogram mode scheme and the list mode scheme are known. In the sinogram mode scheme, the counting circuit 39 performs pulse height discrimination of electrical pulses from each X-ray detector 15, and counts the number of electrical pulses in each of preset energy bands as the number of X-ray photons for each X-ray detector 15. The plurality of energy bands have been set via an input unit 71. In the list mode scheme, the counting circuit 39 performs pulse height discrimination of electrical pulses from each X-ray detector 15, and records the pulse height value of each electrical pulse as the energy value of each X-ray photon in association with the detection time. The counting circuit 39 refers to the record to classify X-ray photons into a plurality of predetermined energy bands and count the number of X-ray photons in each of the plurality of energy bands for each view. The count number data are supplied to the preprocessor 75.

The preprocessor 75 preprocesses the count number data for each energy band supplied from the counting circuit 39. For example, preprocessing includes integral processing of the numbers of photons, logarithmic conversion, X-ray intensity correction, and offset correction.

The reconstruction unit 77 generates a photon counting CT image expressing the spatial distribution of CT values concerning a visualization target energy band of a plurality of energy bands by applying an image reconstruction algorithm to the count number data obtained by preprocessing for the visualization target energy band.

The imaging controller 79 synchronously controls a gate controller 59, an X-ray controller 61, a filter drive controller 63, a collimator drive controller 65, and the counting circuit 39. As in the first embodiment, the imaging controller 79 synchronously outputs commands to the gate controller 59 and the X-ray controller 61 to switch an X-ray source 11 as an X-ray generation target in synchronism with the switching timing of a view. Since the operations of the gate controller 59 and the X-ray controller 61 are the same as those in the first embodiment, a description of them will be omitted. In addition, as in the first embodiment, the imaging controller 79 synchronously outputs commands to the filter drive controller 63 and the collimator drive controller 65 so as to position a wedge filter 21 in front of the X-ray source 11 as an X-ray generation target and position a post-collimator 27 in front of the X-ray detector 15 located on the opposite side of a rotation axis Z to the X-ray source 11. Since the operations of the filter drive controller 63 and the collimator drive controller 65 are the same as those in the first embodiment, a description of them will be omitted. Furthermore, the imaging controller 79 controls the counting circuit 39 so as to read out an electrical signal from the X-ray detector 15 in synchronism with the switching timing of a view. Since the switching timing of a view is the same as that in the first embodiment, a description of it will be omitted.

The second embodiment can therefore provide a photon counting CT apparatus which can execute high-speed imaging. In addition, as compared with the X-ray computed tomography apparatus according to the first embodiment, the photon counting CT apparatus according to the second embodiment can reduce the exposure dose of an subject S by photon counting CT.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray computed tomography apparatus comprising:
an X-ray source ring including a plurality of X-ray sources arrayed circumferentially, the plurality of X-ray sources individually generating X-rays;
an X-ray detector configured to detect X-rays from the X-ray source ring;
a filter support mechanism configured to support at least one wedge filter rotatable about a rotation axis, the at least one wedge filter provided on an inner circumferential side of the X-ray source ring;
a filter driver configured to drive the filter support mechanism;
a controller configured to control the filter driver to rotate the at least one wedge filter about the rotation axis in synchronism with generation of X-rays from the plurality of X-ray sources;
a data collection unit configured to collect digital data corresponding to an intensity of the detected X-rays; and
a reconstruction unit configured to reconstruct a CT image based on the digital data.

2. The X-ray computed tomography apparatus of claim 1, wherein the X-ray source ring comprises, as the plurality of X-ray sources,
a plurality of electron emission sources arrayed circumferentially,
a plurality of gate electrodes configured to apply electric fields to the plurality of electron emission sources, and
an anode configured to generate X-rays upon receiving electrons generated from the plurality of electron emission sources upon receiving electric fields from the plurality of gate electrodes.

3. The X-ray computed tomography apparatus of claim 2, further comprising a gate electrode driver configured to individually drive the plurality of gate electrodes,
wherein the controller synchronously controls the filter driver and the gate electrode driver to position the at least one wedge filter in front of an X-ray source, of the plurality of X-ray sources, which is an X-ray generation target.

4. The X-ray computed tomography apparatus of claim 3, wherein the controller controls the gate electrode driver to circumferentially sequentially cause the plurality of X-ray sources to generate X-rays.

5. The X-ray computed tomography apparatus of claim 3, wherein the controller controls the gate electrode driver to cause a predetermined number of not less than two X-ray sources of the plurality of X-ray sources to generate X-rays.

6. The X-ray computed tomography apparatus of claim 5, wherein the predetermined number is four.

7. The X-ray computed tomography apparatus of claim 2, further comprising a high voltage generation unit configured to generate a high voltage to be applied between the plurality of electron emission sources and the anode.

8. The X-ray computed tomography apparatus of claim 1, wherein the filter support mechanism supports a plurality of wedge filters arrayed circumferentially as the at least one wedge filter, and
the plurality of wedge filters are formed from materials having different X-ray attenuation coefficients.

9. The X-ray computed tomography apparatus of claim 2, further comprising:
a gate electrode driver configured to individually drive the plurality of gate electrodes;
a collimator support mechanism provided on an inner circumferential side of the X-ray detection ring and configured to support at least one post-collimator rotatable about the rotation axis; and
a collimator driver configured to drive the collimator support mechanism,
wherein the controller synchronously controls the filter driver, the collimator driver, and the gate electrode driver to position the at least one wedge filter in front of an X-ray source, of the plurality of X-ray sources, which is an X-ray generation target, and position the at least one post-collimator in front of an X-ray detector facing the X-ray source as the X-ray generation target.

10. The X-ray computed tomography apparatus of claim 9, wherein the collimator support mechanism supports a plurality of post-collimators arrayed circumferentially as the at least one post-collimator, and
the plurality of post-collimators are formed from materials having different X-ray attenuation coefficients.

11. The X-ray computed tomography apparatus of claim 1, further comprising an X-ray detection ring provided next to the X-ray source ring and including the plurality of X-ray detectors arrayed circumferentially.

12. An X-ray computed tomography apparatus comprising:
an X-ray source ring including a plurality of X-ray sources arrayed circumferentially, the plurality of X-ray sources individually generating X-rays;
an X-ray detector configured to detect X-rays from the X-ray source ring;
a collimator support mechanism provided on an inner circumferential side of the X-ray detection ring and configured to support at least one post-collimator rotatable about a rotation axis;
a support mechanism driver configured to drive the collimator support mechanism;
a controller configured to control the support mechanism driver to rotate the at least one post-collimator about the rotation axis in synchronism with generation of X-rays from the plurality of X-ray sources;
a data collection unit configured to collect digital data corresponding to an intensity of the detected X-rays; and
a reconstruction unit configured to reconstruct a CT image based on the digital data.

13. A photon counting CT apparatus comprising:
an X-ray source ring including a plurality of X-ray sources arrayed circumferentially, the plurality of X-ray sources individually generating X-rays;
an X-ray detector configured to detect X-rays from the X-ray source ring;
a filter support mechanism configured to support at least one wedge filter rotatable about a rotation axis, the at least one wedge filter provided on an inner circumferential side of the X-ray source ring;
a filter driver configured to drive the filter support mechanism;
a controller configured to control the filter driver so as to rotate the at least one wedge filter about the rotation axis in synchronism with generation of X-rays from the plurality of X-ray sources;
a counting unit configured to count the number of detected X-ray photons; and
a reconstruction unit configured to reconstruct a photon counting CT image based on the number of photons.

14. The photon counting CT apparatus of claim 13, wherein the X-ray source ring comprises, as the plurality of X-ray sources,
a plurality of electron emission sources arrayed circumferentially,
a plurality of gate electrodes configured to apply electric fields to the plurality of electron emission sources, and
an anode configured to generate X-rays upon receiving electrons generated from the plurality of electron emission sources upon receiving electric fields from the plurality of gate electrodes.

15. The photon counting CT apparatus of claim 14, further comprising a gate electrode driver configured to individually drive the plurality of gate electrodes,
wherein the controller synchronously controls the filter driver and the gate electrode driver to position the at least one wedge filter in front of an X-ray source, of the plurality of X-ray sources, which is an X-ray generation target.

16. The photon counting CT apparatus of claim 14, wherein the controller controls the gate electrode driver to circumferentially sequentially cause the plurality of X-ray sources to generate X-rays.

17. The photon counting CT apparatus of claim 14, wherein the controller controls the gate electrode driver to cause a predetermined number of not less than two X-ray sources of the plurality of X-ray sources to generate X-ray photons.

18. The photon counting CT apparatus of claim 17, wherein the predetermined number is four.

19. The photon counting CT apparatus of claim 14, further comprising a high voltage generation unit configured to generate a high voltage to be applied between the plurality of electron emission sources and the anode.

20. The photon counting CT apparatus of claim 13, wherein the filter support mechanism supports a plurality of wedge filters arrayed circumferentially as the at least one wedge filter.

21. The photon counting CT apparatus of claim 14, further comprising:
a collimator support mechanism configured to support at least one post-collimator so as to make the post-collimator rotatable about the rotation axis; and
a collimator driver configured to drive the collimator support mechanism,
wherein the controller synchronously controls the filter driver, the collimator driver, and the gate electrode driver to position the at least one wedge filter in front of an X-ray source, of the plurality of X-ray sources, which is an X-ray generation target, and position the at least one post-collimator in front of an X-ray detector facing the X-ray source as the X-ray generation target.

22. The photon counting CT apparatus of claim 21, wherein the collimator support mechanism supports a plurality of post-collimators arrayed circumferentially as the at least one post-collimator.

23. The photon counting CT apparatus of claim 13, further comprising an X-ray detection ring provided next to the X-ray source ring and including the plurality of X-ray detectors arrayed circumferentially.

24. An photon counting CT apparatus comprising:
an X-ray source ring including a plurality of X-ray sources arrayed circumferentially, the plurality of X-ray sources individually generating X-rays;
an X-ray detector configured to detect X-rays from the X-ray source ring;
a collimator support mechanism provided on an inner circumferential side of the X-ray detection ring and configured to support at least one post-collimator rotatable about a rotation axis;
a support mechanism driver configured to drive the collimator support mechanism;
a controller configured to control the support mechanism driver to rotate the at least one post-collimator about the rotation axis in synchronism with generation of X-rays from the plurality of X-ray sources;
a counting unit configured to count the number of detected X-ray photons; and
a reconstruction unit configured to reconstruct a photon counting CT image based on the number of photons.
